(19) United States
(12) Patent Application Publication
(10) Pub. No.: US 2025/0195274 A1
(43) Pub. Date: Jun. 19, 2025

(54) LASER PHACOLYSIS SYSTEM AND METHOD

(71) Applicant: UNIVERSITY OF SOUTH FLORIDA, Tampa, FL (US)

(72) Inventors: Ramesh S. Ayyala, Tampa, FL (US); Mitchell Robert Harrah, Tampa, FL (US); Abdul Mohaimen Safi, Tampa, FL (US); Ashwin Bharadwaj Parthasarathy, Tampa, FL (US); Sadhu Moka, Tampa, FL (US)

(21) Appl. No.: 18/987,416

(22) Filed: Dec. 19, 2024

Related U.S. Application Data
(60) Provisional application No. 63/611,953, filed on Dec. 19, 2023.

(51) Int. Cl. A61F 9/008

(52) U.S. Cl. CPC A61F 9/00814; A61F 2009/0087; A61F 2009/00887

(57) ABSTRACT

Methodology of phacoemulsification of a biological target such as a cataract with the use of a system including a laser diode configured to generate light in a millisecond pulsed regime and an optical fiber, while avoiding thermal diffusion of heat throughout the target. The used light has a wavelength within an absorption band of water and an average power within a milliwatt range; optionally, the output surface/facet of the optical fiber is curved and/or equipped with a termination configured to spatially converge output light upon propagating of such light through the termination.

### CROSS-REFERENCE TO RELATED APPLICATIONS

This US Patent application claims priority from and benefit of the U.S. Provisional Patent Application No. 63/611,953 filed on Dec. 19, 2023, the disclosure of which is incorporated by reference herein.

### TECHNICAL FIELD

The present invention relates generally to a methodology for phacoemulsification and, more particularly, to a methodology of purposefully carrying the process of phacolysis at the very wavelength(s) that approximately equal(s) to those at which absorption of light by an eye is substantial (and, for that reason, which wavelengths are avoided by related art) to facilitate the reduction of costs of the procedure.

### RELATED ART

Conventionally and currently, phacoemulsification is aided mainly by a femtosecond pulse type laser source (for example, with the use of a neodymium-doped yttrium aluminum garnet (Nd: YAG) crystal laser), which is widely recognized as a very expensive proposition.

With high costs, low accessibility, and high utility requirements of femtosecond phacolysis devices, many rural areas and/or developing countries have resorted to outdated techniques of intracapsular cataract extraction and extracapsular cataract extraction. First, such intracapsular cataract extraction procedure involves the removal of the entire lens capsule and surrounding parts-such removal, unfortunately, leads to multiple complications both during and after surgery. A related methodology—the extracapsular cataract extraction-works similarly to phacoemulsification, except that the cataract is removed as a solid and not emulsified. This results in larger incisions in the eye and a high occurrence of astigmatism, or blurry vision, after the surgery. (See, for example, Ruit, S., et al., Low-cost high-volume extracapsular cataract extraction with posterior chamber intraocular lens implantation, in Nepal. Ophthalmology, 1887-1892; 1999.) With the many complications of these outdated procedures, the need remains strong in a low-cost, portable phacolysis-related solution.

### SUMMARY OF THE INVENTION

Embodiments of the invention provide a method that includes a step of delivering light, generated by a laser diode source of light, to a target through a first medium surrounding the target (while not interacting the light with the first medium) along at least a first portion of an optical path of the light from the laser diode (the first portion of the optical path traversing the first medium), a step of irradiating a target with the light that is configured to ensure that heating of the target, caused by the light, is substantially confined to an irradiated area of the target and not lost by thermal diffusion through the target (or, not thermally diffused through the target), and a step of thermally emulsifying or liquifying the irradiated area of the target with the light. The generation of such light is optionally performed in a pulsed regime with a duration of a pulse being not shorter than a millisecond and an average power within a milliwatt range; the process of delivering may include propagating the light along at least the first portion of the optical path while not changing a degree of divergence or a degree of convergence of the light during the propagating.

Embodiments also provide an apparatus that is configured as a laser phacolysis apparatus and that includes a laser diode configured to generate light in a millisecond pulsed regime, and an optical fiber element structured to be cooperated with the laser diode at a proximal end of the optical fiber element to receive the light from the laser diode—as well as a method for using such apparatus.

Embodiments further provide a method that includes carrying a laser phacolysis procedure without the use of a femtosecond laser source (and/or without traversing laser light through the iris and/or without forming an incision at the cornea) by performing at least the following steps: delivering pulsed laser light with a millisecond pulse duration from a laser diode source to the lens of an eye through the sclera while not interacting said pulsed laser light with the sclera and with at least a portion of a humor body of the eye along a path of said delivering, and ablating at least a portion of the lens of the eye while not heating the cornea above 65 degrees Centigrade. Optionally, such pulsed laser light is configured to ensure that heating of the lens, caused by the light, is substantially confined to an irradiated area of the lens and not lost by thermal diffusion through the target (or, not thermally diffused through the target).

### BRIEF DESCRIPTION OF THE DRAWINGS

The invention will be more fully understood by referring to the following Detailed Description of Specific Embodiments in conjunction with the Drawings, of which:

FIG. 1 provides a schematic of the apparatus structured according to one embodiment of the invention.

FIG. 2 illustrates an axisymmetric slice of the ocular model, as discussed below.

FIG. 3: Photograph Scale of the Formulations Used for Gelatin Experiments

FIG. 4: LOCS III Classification of Phantom Gel Cataract Samples of Varying Masses Per 750 mL of Water.

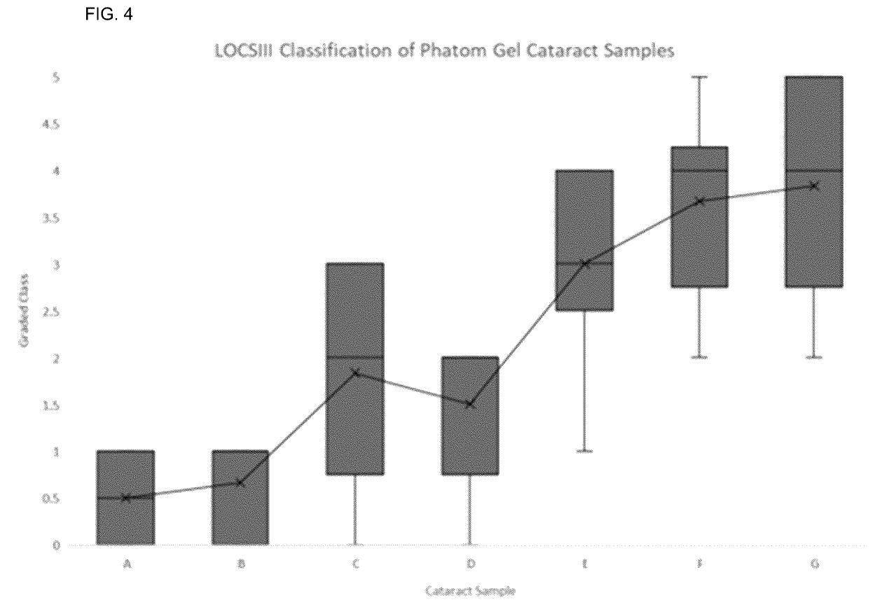

FIG. 5 illustrates area of Thermal Damage on Less Opaque Gelatin Surfaces.

FIG. 6 illustrates area of Thermal Damage on More Opaque Gelatin Surfaces.

FIG. 7 illustrates a specific embodiment of a optical fiber-based component used to deliver irradiating light to the target.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another.

### DETAILED DESCRIPTION

It is well recognized that cataracts are an emerging public health problem for a population that is rapidly aging. The Centers for Disease Control and Prevention (CDC) estimated that over 30 million patients were affected by cataracts in 2020, in the United States (Centers for Disease Control and Prevention, 2022). In simple terms, cataracts occur when fibers of an older natural eye lens solidify to create an opaque solid at the optical center of the ocular lens that creates symptoms such as blurry vision, astigmatism, and even blindness. Clinically, cataracts present as blurry vision in preliminary stages, leading to blindness if untreated. This results in a massive loss in a patient's quality of life, affecting their day-to-day activities and their ability to participate in many societal functions.

Currently, the most common and effective treatment for cataracts involves either a low tech version of extracapsular cataract extraction that requires large incision (sized at about 5-7 mm) and closure with sutures and longer period to heal—and occurrence of high astigmatism, or blurry vision, after the surgery; or, alternatively, a small incision cataract surgery using phacoemulsification—that is, a surgical procedure that facilitates removal of the cataract through a 1.5 to 2.6 mm incision and that can be suture-less and faster healing (in comparison with the extraction-based treatment).

Phacoemulsification is a modern cataract surgery method in which the eye's internal lens is usually emulsified with an ultrasonic handpiece and aspirated from the eye with the help of an operating microscope. Aspirated fluids are replaced with irrigation of balanced salt solution to maintain the anterior chamber followed by intraocular lens implantation. More recently, optical methods such as femtosecond lasers have been used to perform certain steps of the cataract surgery including astigmatic correction, entry into the eye and cutting the capsular bag and slight softening of the hard nucleus. Here, the primarily used source of light is a Nd: YAG crystal-based laser source, used to create femtosecond pulses at about 1064 nm to heat and emulsify the cataract portion. The person of skill in the art will readily appreciate that one of the compelling reasons to utilize the Ng-YAG based laser source is explained by the conventional desire to stay away from the wavelengths at which the eye tissue has high absorption—in order to not damage the eye tissue when irradiating the cataract therethrough.

However—as well recognized in related art—the used femtosecond laser is not only (sometimes prohibitively) expensive but can achieve only minimal softening of the hard nucleus, thereby often causing the need for additional use of conventional ultrasonic energy to complete the liquification. Further, the required equipment takes up about two square meters of floor space, thereby understandably reducing the portability of such equipment between surgical suites and medical centers.

With that in mind, the cost of cataract surgery in many developing countries and rural areas can reach sometimes thousand(s) of dollars—which substantially exceeds a typical annual income in such areas (see, for example, Liu, Y.-C., et al., Cataracts, The Lancet, 600-612, 2017) while the lack of portability creates multiple logistic hurdles that prospective patients must endure such as requiring travel to the nearest medical center with the technology, and finding an affordable place to stay during initial recovery, as well as travel/stay for post-operative care.

In accordance with implementation(s) of the idea of the present invention, the use of a first-of-its-kind millisecond laser phacoemulsification prototype is disclosed, the operation of which is turning on the use of thermal energy within a contained area, thereby facilitating emulsification of the cataract and lens media while keep the operation substantially free of damage to surrounding optical tissue. Persisting shortcomings that are associated with phacoemulsification of the cataractous lens currently performed with a femtosecond laser source and that stem from the need to avoid light at wavelengths at which absorption of light at the eye is substantial (thereby necessarily and inevitably leading to the use of bulky and costly equipment) are solved by a novel laser phacolysis methodology that does not utilize the femtosecond laser source. The proposed methodology employs shifting the operational wavelength of used light to that falling within the band of absorption of water while, at the same time, delivering such used light to the cataract under conditions that substantially prevent the used light from interacting with a portion of the eye outside and/or surrounding the cataractous lens.

In stark contradistinction with the conventional related art, the provided solution enables the use of a low-cost off—the shelf laser diode sources for efficient implementation of the laser phacolysis procedure.

One of the main features of the proposed approach is the devising of millisecond laser pulses that result in production of localized heating of the cataract and lead to phacolysis. It has been realized that, for successful thermal phacolysis, the laser pulse width needs to be smaller than the thermal relaxation time of the tissue: such relationship ensures that the heat generated by the laser is confined to the illumination spot and is not lost by thermal diffusion. By selecting millisecond-duration light pulses at a wavelength at which absorption of light in the eye is substantial (for example, light from a diode laser at 1480 nm), the embodiment of the invention targets a water absorption peak to result in increase in tissue absorption coefficient by at least an order of magnitude (as well as decrease in relaxation times by at least two orders of magnitude) as compared to currently used femtosecond laser pulse-based methodology at 1064 nm of related art. The selection of wavelength also permits more efficient mechanical stress confinement by 50%, improving ablation efficiency. This produces stark operational advantages over the use of a femtosecond-laser-pulse based phacolysis technique.

The proposed method of delivery of laser light facilitates raising temperatures of the cataracts in the eye to a level that is sufficient to emulsify the cataracts but without damaging any surrounding tissues. In particular—as will be understood from the discussion below—an embodiment of the proposed approach may include (i) delivering light, generated by a laser diode source of light, to a target through a first medium (biological tissue) surrounding the target (a portion of the natural lens of an eye) while not interacting the light with the first medium (and/or not producing any thermal effects in the first medium) along at least a first portion of an optical path of the light from the laser diode (here, the first portion of the optical path traverses the first medium); (ii) irradiating a target with the light (which is configured such as to ensure that heating of the target, caused by the light, is substantially confined to an irradiated area of the target and not lost by thermal diffusion through the target; and (iii) thermally emulsifying or liquifying the irradiated area of the target with the light.

The idea of the invention stems from the realization that laser phacolysis can be achieved with the use of millisecond pulses to achieve ablation (˜ liquification, emulsification) of the cataract.

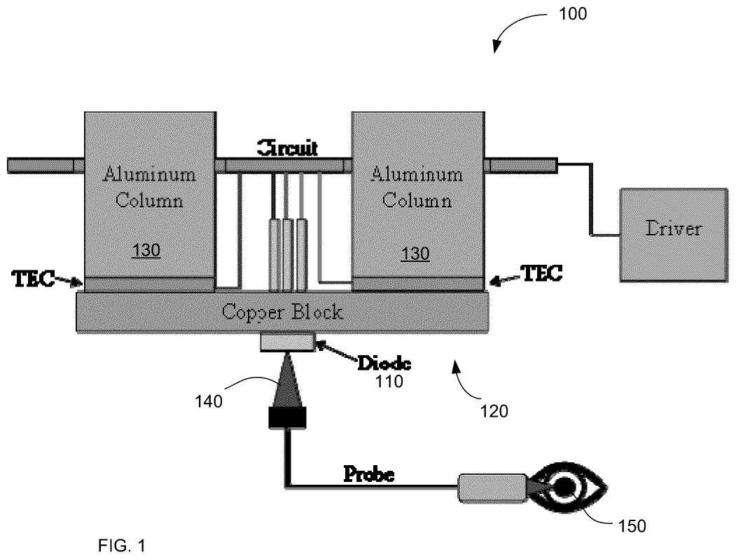

FIG. 1 schematically illustrates a custom low-cost laser phacolysis instrument 100, fabricated with the use of a high-power pulsed laser diode 110. In this example, the apparatus 100 features a 1480 nm 2000 mW laser diode (Thorlabs L1480G1; attached to a custom laser mount 120) that was intentionally, purposefully selected to spectrally substantially match one of the water absorption peaks in the infrared portion of the spectrum. Laser mount includes a copper block and has two thermoelectric cooling (TEC) elements attached to it. Two solid aluminum columns 130 were attached to the copper block to further facilitate cooling of the diode 110. Additionally, each column 130 had three thermal fins to help with heat dissipation. Here, the thermal fins were added to the three sides of the cooling columns that are not facing each other. The addition of thermal fins helped the cooling columns add more surface area to increase conduction-based cooling, which in turn helped disperse the heat across a more considerable amount of space. However, when adding thermal fins, it is essential to analyze the overall performance of the fins to ensure that proper cooling can take place. For the prototype, the Awxlumv Heat Sink 40×40×11 mm Aluminum Small Heatsinks were used.

The entire arrangement was secured in a box, with four high speed fans. The laser 110 and the TEC elements were connected to a custom printed circuit board, with signal conditioning components, and connected to an external laser diode controller (Thorlabs, ITC4020). In operation, the laser diode controller electronically pulsed the laser at a user-defined frequency and duty cycle to ensure generation of light (the optical output 140) in a pulsed regime with a duration of a pulse being not shorter than a millisecond and an average power in a milliwatt range.

Finally, the optical output 140 of the laser diode 110 was collimated and appropriately coupled to a custom fiber optic probe (Fiberoptic Systems Inc.) for illuminating of the target 150 (the cataract in the eye) as intended. In one specific case, for example, the optical fiber based probe was inserted in the lateral side of the sclera via a small incision. The laser source 110 was operated to produce the pulsed output 140 to induce thermal and mechanical changes to the material of the target 150 resulting in the emulsification of the target. (It is understood, therefore, that the delivery of light to the target included propagating such light along at least the first portion of the optical path of light while not changing a degree of divergence or a degree of convergence of the light during such propagating towards the target; and/or the delivery of light to the target included delivering such light through a second medium that was different from the first medium.) Following the emulsification, the cataract target 150 was removed via a small vacuum probe and the incision(s) were resealed.

The cataract emulsification was the result of the thermal interaction of laser pulses with the cataract. One primary consideration is the duration of the pulse (ON time t_{ON}). Ablation of the cataract occurs when the ON time t_{ON}<tᵣ, where tᵣ is the critical relaxation time of the cataract given

$${t}_{r} = \frac{1}{κ ( 3 {μ}_{a} ( {μ}_{a} + {μ}_{s}^{′} ) )} .$$

Here, μ_{α} and μ′ₛ are the absorption and reduced scattering coefficients of the cataract, and x is the thermal diffusivity given by

$$κ = \frac{K}{{C}_{p} ρ} ,$$

where K is the thermal conductivity, Cₚ is the specific heat at constant pressure and ρ is the density of the cataract. Further, the efficiency of the ablation process can be modeled using a mechanical relaxation time.

$${t}_{m} = \frac{1}{v ( 3 {μ}_{a} ( {μ}_{a} + {μ}_{s}^{′} ) )}$$

is used, where v is the speed of sound within the material. For efficient ablation the mechanical stress created in the cataract should be maximized. Mechanical stress is given by σₚ=AΓμ_{α}Φ₀, where σₚ is the peak thermoelastic stress, Γ is the Grüneisen coefficient, and Φ₀ is the incident radiant exposure. A is the stress confinement factor, given by

$$A = \frac{1 - {e}^{- \frac{{t}_{p}}{{t}_{m}}}}{\frac{{t}_{p}}{{t}_{m}}} .$$

The product Aμ_{α} determines the mechanical efficiency of ablation; this product is 1.5× higher at 1480 nm compared to 1064 nm. Table 1 below summarizes the values used to calculate a critical relaxation time of tᵣ=0.83 s and tₘ=2.86*10⁻⁷s for a 1480 nm wavelength laser-based illumination.

TABLE 1

| Property | | |
|---|---|---|
| (Units) | Notation | Value |
| Density | ρ | 1000 |
| (kg/m³) | | |
| Thermal | K | 0.4 |
| Conductivity | | |
| (W/(m*K)) | | |
| Specific Heat | Cₚ | 3000 |
| (J/(kg*K)) | | |
| Absorption | μₐ | 10 |
| Coefficient at | | |
| 1480 nm | | |
| (1/cm) | | |
| Reduced | μₛ′ | 20 |
| Scattering | | |
| Coefficient at | | |
| 1480 nm | | |
| (1/cm) | | |
| Penetration | δ | 0.03 |
| Depth (cm) | | |
| Thermal | κ | 0.0013 |
| Diffusivity | | |
| (cm²/s) | | |
| Critical | tᵣ | 0.83 |
| Relaxation | | |
| Time (s) | | |

### Simulation.

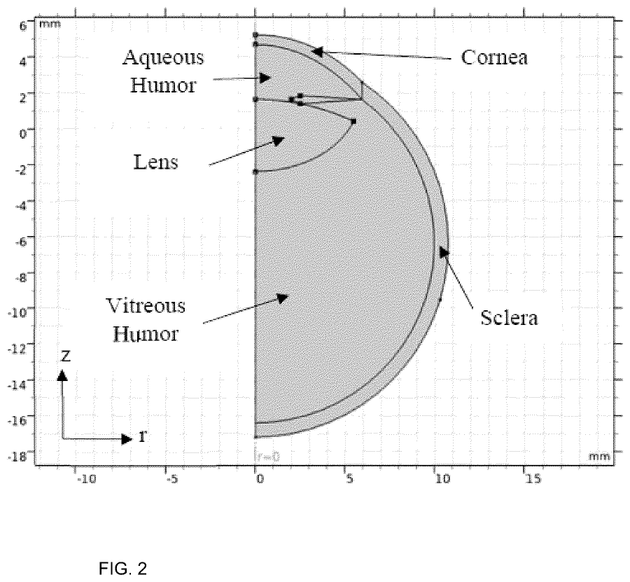

A COMSOL simulation of light-matter thermal interaction in the cataract/lens was performed to verify that the laser diode light output operating at millisecond pulses could, indeed, cause phacolysis without damaging the surrounding eye tissue. The sclera of the eye was modeled as a perfect circle with a thickness of 0.96 mm. The shape of the cornea and lens were modeled using the equation, r∧2+(1+Q)z∧2−2zR=0, where r is radial position on the curve, Q is the asphericity, z is the position on the z-axis, and R is the maximum radius of the curve. The contribution of the fovea was substantially ignored, and the iris was simplified and considered to have a diamond shape and a maximum thickness of 0.46 mm. Finally, a 3D model was generated assuming that eye was axisymmetric. Table 2 below in conjunction with the schematic of FIG. 2 below display properties used to simulate shapes of the lens and cornea (with positive or negative orientations denoting if a given curve is concave or convex).

TABLE 2
Properties used to simulate ocular structure

| | | | Apex | |
|---|---|---|---|---|
| | | Radius | Location | |
| Curve | Asphericity | [mm] | [mm] | Orientation |
| Anterior | −0.94 | 12.40 | 1.59 | Negative |
| Lens | | | | |
| Posterior | 0.96 | 8.10 | −2.59 | Positive |
| Lens | | | | |
| Anterior | −0.18 | 7.77 | 5.19 | Negative |
| Cornea | | | | |
| Posterior | −0.60 | 6.40 | 4.64 | Negative |
| Cornea | | | | |

Table 3 displays the thermal properties of the different tissue types in the eye. (according to Mirnezami, S. A., et al., Temperature Distribution Simulation of the Human Eye Exposed to Laser Radiation. Journal of Lasers in Medical Sciences, 175-181, 2013). Here, it was assumed that the cataract filled the lens entirely, and that aqueous humor was the only component that changed its density with temperature. The density of the aqueous humor (Table 4) was modeled as ρ=ρ₀[1−β(T−T₀)]. Here, ρ₀ represents the reference density in kg/m³, β is the volume expansion coefficient in K⁻¹, and T₀ is the reference temperature in ° C. Given that the thermal interactions on the cataract was the only point of interest, we have selected to only define the solid material properties of the cataract and none of the other solid media in the ocular region.

TABLE 3
Selected Properties of Ocular Biological Materials

| | | | Heat |
|---|---|---|---|
| | | | Capacity at |
| | | Thermal | Constant |
| | Density | Conductivity | Pressure |
| Material | (kg/m³) | (W/(m*K)) | (J/(kg*K)) |
| Cornea | 1050 | 0.58 | 3500 |
| Aqueous | Eq 3.4 | 0.58 | 4200 |
| Humor | | | |
| Lens | 1050 | 0.40 | 4200 |
| Vitreous | 1050 | 0.603 | 4200 |
| Humor | | | |
| Sclera | 1050 | 1.0042 | 3500 |
| Iris | 1050 | 1.0042 | 3500 |

TABLE 4
Aqueous Humor Material Property Values

| Property (Units) | Value |
|---|---|
| Reference Density (kg/m³) | 996 |
| Volume Expansion Coefficient (K⁻¹) | 0.0003 |
| Reference Temperature (° C.) | 34 |
| Dynamic Viscosity (N*s/m²) | 0.00074 |
| Specific Heat Ratio (1) | 1.66 |

Table 5 displays the mechanical properties of the different tissue types in the eye. Here, the absorption coefficient of the cataract was selected at a wavelength of 1485 nm (Belikov, A. V., et al., Optical Properties of Human Eye Cataractous Lens in vitro in the Visible and Near-IR Ranges of the Spectrum, Optics and Spectroscopy, 574-579, 2019)

TABLE 5
Cataract Lens Material Property Values

| Property (Units) | Value |
|---|---|
| Absorption Coefficient (cm⁻¹) | 10 |
| Young's Modulus (kPa) | 6000 |
| Poisson's Ratio | 0.47 |
| Volume Expansion Coefficient (K⁻¹) | 0.0003 |

The primary objectives of the performed COMSOL simulation were to assess the maximum temperatures and stress at the optical center of the lens and any tissue damage that occurs on the cornea, for different values of pulse width and power. Optical, thermal, and mechanical effects of laser irradiation on the cataract were performed for a duration of 120s using the Bioheat Transfer, Solid Mechanics, Radiative Beam in Absorbing Medium, and Laminar Flow physics modules and the Thermal Expansion, Heat Transfer with Radiative Beam, and Nonisothermal Flow Multiphysics modules. The simulation began with the laser being ‘ON’ for ten seconds. The laser was applied in the negative z-direction, pulsing at the specified pulse width interval.

Thermal damage to the corneal tissue was modeled using

$$\frac{\partial α}{\partial t} = \frac{1}{{t}_{d , h}} ( T > {T}_{d , h} ) .$$

Here α is the damage percentage, t_{d,h} is the amount of time tissue spends at a temperature above the damage temperature T_{d,h}. The benchmark damage temperature was 59° C., and the damage time was 60 sec. Further, if the corneal tissue reached 65° C., the heated corneal tissue was considered to be damaged.

### Tissue Simulating Phantom.

Experimental validation of the laser system was performed using tissue simulating gelatin phantoms. Multiple gelatin phantoms were fabricated to simulate different grades of cataracts using methods described elsewhere (Farrer, et al., 2015), with gelatin concentrations defined in Table 6.

TABLE 6
Accepted Concentrations from the Survey Performed

| | Mass of | Mass of | Mass of |
|---|---|---|---|
| | Gelatin per | Gelatin per | Gelatin per |
| Formulation | 750 mL of | 1 mL of | 50 mL of |
| Label | Water (g) | Water (g) | Water (g) |
| A | 25.00 | 0.03 | 1.67 |
| B | 50.00 | 0.07 | 3.33 |
| C | 111.00 | 0.15 | 7.40 |
| D | 166.50 | 0.22 | 11.10 |
| E | 222.00 | 0.30 | 14.80 |
| F | 277.50 | 0.37 | 18.50 |
| G | 333.00 | 0.44 | 22.20 |

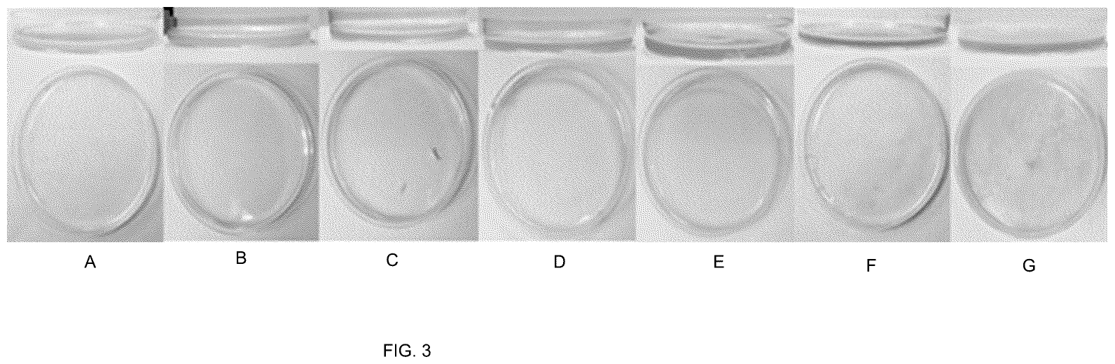

Here, the desired mass of gelatin (Sigma-Aldrich, G6144, Type A, 90-110 g Bloom) was dispersed in 50 ml of tap water that had been brought to a boil. Once the water began to boil rapidly, the gelatin powder was slowly added to the heated solution, while the solution was undergoing rigorous mixing, to avoid the gelatin forming large solid particles. After the mixing, the solution was poured into petri dishes to a height of five millimeters which matches the approximate thickness of ocular lenses. The samples were refrigerated for twenty-four hours, and images of each sample were taken for a survey, see FIG. 3.

### Classification of Tissue Simulating Phantoms.

Cataracts were classified by using the Lens Opacity Classification System III (LOCSIII, as known in related art). Six ophthalmologists individually rated the gelatin phantoms according to the Lens Opacities Classification System III (LOCS III), based on randomly presented pictures of the phantoms' top- and side-views. The result of this rating is presented in Figure XX, showing a sigmoidal curve, which flattens out at around grade 4.

### Experimental Methods.

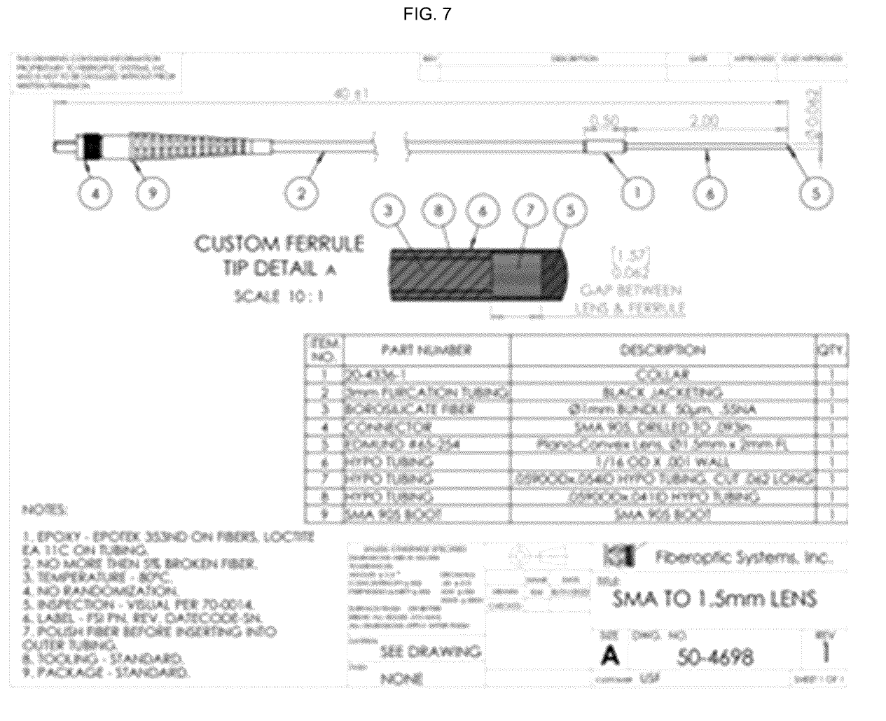

Example of a specialized fiber-optic probe is illustrated in FIG. 7. Here, For this device, a specialized probe has been constructed to meet the surgical requirements for phacolysis, while maintaining the power and pulses of the laser beam. Given the borosilicate fiber and tubing specifications, the fiber will maintain the high power that is delivered into the cataract. Further, the Hypo Tubing was a medical-grade stainless steel ending of the fiber with a design dedicated to surgical and medical imaging applications. The SMA 905 connection at the other end is to connect directly into the custom-made mount.

For the tissue phantom validation experiments, the laser diode system was operated at 1 W at 10, 30, 50, 70, and 90 ms pulse widths, each with a 50% duty cycle, as well as for continuous wave (CW) illumination. The pulse widths were selected to match those used in the COMSOL simulation. The laser was positioned to illuminate the surface of the gelatin phantom at a fixed position until the gelatin liquefied for up to a maximum of 5 minutes. An image of the surface of the phantom was recorded using a digital camera. The time to liquification was measured, and the area of the liquid pool was estimated using a custom algorithm implemented in MATLAB. This procedure was repeated 5 times for each phantom and pulse width. A Wilcoxon rank-sum test was used to determine if the resulting damage areas were statistically different for each phantom/pulse width.

### Results.

### (1) Simulation Results on Pulse Width

The question posed with the COMSOL simulations concerned the magnitude and extent of temperature increases in the lens due to laser irradiation. Table 7 presents maximum temperature reached at the refractive center of the lens for various laser illumination powers and pulse widths. The radius of the laser was fixed at 287 μm to match the optical probe. In general, higher irradiation powers cause larger temperature increases. Critically, for every irradiation power, pulsed operation results in lower temperature increases compared to continuous wave operation.

TABLE 7
Maximum Temperatures at the Refractive Center of the Human
Lens under Various Pulse Widths and Laser Radius of 287 μm

| Power | Max Temperature (° C.) at | | | | | |
|---|---|---|---|---|---|---|
| (mW) | Continuous | 10 ms | 30 ms | 50 ms | 70 ms | 90 ms |
| 500 | 110.54 | 75.89 | 76.36 | 76.83 | 77.35 | 78.01 |
| 1000 | 182.17 | 111.49 | 112.40 | 113.40 | 114.46 | 115.60 |
| 1500 | 257.80 | 147.43 | 148.80 | 150.30 | 151.90 | 153.59 |
| 2000 | 343.17 | 183.53 | 185.34 | 187.26 | 189.37 | 192.00 |
| 2500 | 455.32 | 220.48 | 222.74 | 225.24 | 227.87 | 231.13 |
| 3000 | 636.95 | 259.03 | 261.72 | 264.68 | 267.83 | 271.80 |

With the temperature data alone, it was problematic to visualize the effect of the heat localization that we would expect from the ablation mentioned earlier. To illustrate this, Table 8 below shows the maximum area of the lens that reaches or exceeds 100 degrees Celsius.

TABLE 8
Area of the Lens Exceeding 100 Degrees Celsius under
Various Pulse Widths and Laser Radius of 287 μm

| Power | Area in Squared Millimeters | | | | | |
|---|---|---|---|---|---|---|
| (mW) | Continuous | 10 ms | 30 ms | 50 ms | 70 ms | 90 ms |
| 500 | .73 | 0.13 | 0.15 | 0.16 | 0.18 | 0.19 |
| 1000 | 1.90 | 0.78 | 0.78 | 0.79 | 0.79 | 0.80 |
| 1500 | 2.89 | 1.37 | 1.37 | 1.38 | 1.38 | 1.39 |
| 2000 | 3.74 | 1.91 | 1.92 | 1.92 | 1.93 | 1.93 |
| 2500 | 4.56 | 2.42 | 2.42 | 2.43 | 2.43 | 2.44 |
| 3000 | 5.36 | 2.89 | 2.89 | 2.89 | 2.90 | 2.91 |

The next set of result, illustrated the extent of thermal damage. However, even with the lowered maximum temperatures, the tissue damage on the posterior corneal lens was still a key point of interest. Given the interest in tissue damage, Table 9 shows whether any tissue damage was seen on the posterior corneal lens boundary.

TABLE 9
Temperature at the posterior corneal surface of the Lens
under Various Pulse Widths and Laser Radius of 287 μm

| Power | Temperature (degC.) at | | | | | |
|---|---|---|---|---|---|---|
| (mW) | Continuous | 10 ms | 30 ms | 50 ms | 70 ms | 90 ms |
| 500 | 48.8 | 42.8 | 42.8 | 42.8 | 42.8 | 42.8 |
| 1000 | 60.93* | 48.92 | 48.93 | 48.92 | 48.95 | 48.96 |
| 1500 | 73.09* | 55.01 | 55.02 | 55.00 | 55.05 | 55.07 |
| 2000 | 84.89* | 61.03* | 61.05* | 61.05* | 61.10* | 61.13* |
| 2500 | 96.82* | 67.02* | 67.06* | 67.05* | 67.11* | 67.17* |
| 3000 | 108.97* | 73.11* | 73.02* | 73.06* | 73.12* | 73.25* |

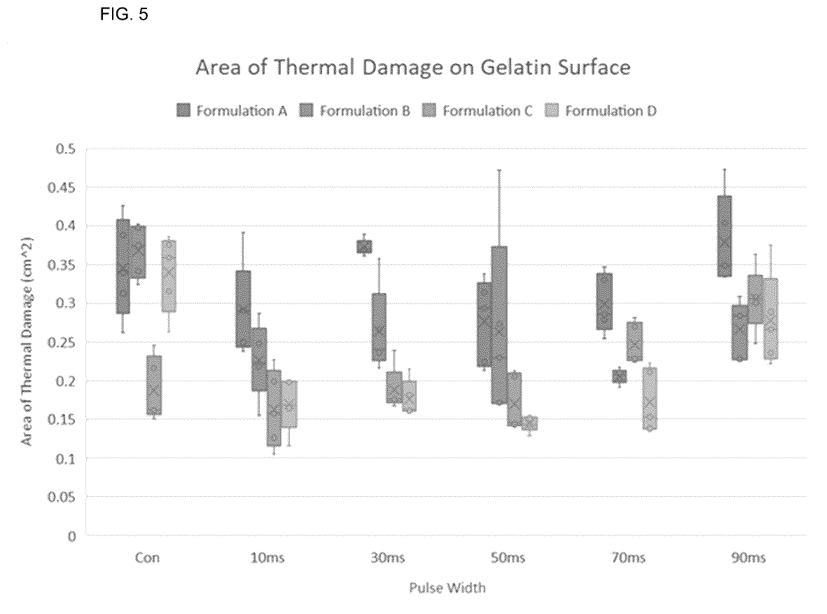

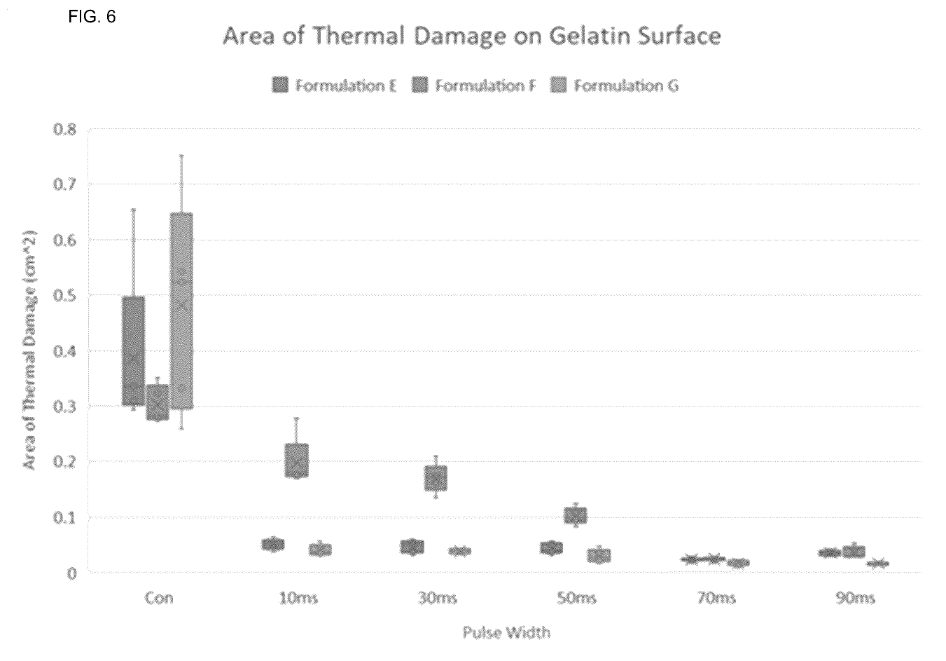

FIGS. 5 and 6 highlight the results of the tissue phantom experiment.

Gelatin Sample Results with a LOCS Grade Less than 2.5

FIG. 5 plots the mean area of thermal damage on the surface of the tissue surface (area of liquefaction) for sample formulations A through D, for different pulse widths. These formulations comprise of gelatin phantoms that were graded to be less than 2.5 on the LOCS III scale. The effect of laser damage on these samples do not present a clear relationship, but some trends are apparent. In most phantoms, the damage due to pulsed illumination was lower than those for continuous illumination. FIG. 5 shows the mean damage area for these phantoms. It is possible that due to the lower melting point of these mixtures, clear features were not visible which made calculation of damage areas with the MATLAB algorithm error prone.

Gelatin Sample Results with a Grade More than 2.5

FIG. 6 and Table 11 show the damage areas recorded for the three highest graded formulations (formulations E thorough G) of gelatin mixtures which represent cataracts graded above 2.5 on the LOCS III scale. In all phantoms, the damage area for continuous illumination was greater that for pulsed illumination with strong statistical significance. Interestingly, the effects did not monotonically increase with phantom grade. The effects on formulation F were weaker than those on formulation G.

TABLE 10
Median Area (cm²) of Present Thermal Damage
on More Opaque Gelatin Surfaces

| | E | F | G |
|---|---|---|---|
| Continuous | 0.335 | 0.283 | 0.523 |
| 10 ms | 0.051* | 0.180* | 0.037* |
| 30 ms | 0.049* | 0.171* | 0.041* |
| 50 ms | 0.040* | 0.098* | 0.027* |
| 70 ms | 0.022* | 0.024* | 0.014* |
| 90 ms | 0.037* | 0.029* | 0.016* |

### DISCUSSION

As the person of skill now readily appreciates, the presented discussion focuses on initial demonstration of the use of a low-cost laser device for phacoemulsification of cataracts. The goal of the presented research was to establish that millisecond laser pulses can produce localized heating of the cataract and lead to phacolysis (while—in at least one specific case—irradiating the target with a non-collimated beam of light emerging from the optical fiber that delivered the light to the target along the path traversing aqueous humor).

Finite element simulations of heat transfer in the eye confirmed that the millisecond pulse laser could successfully increase the temperature in the cataract, while maintaining minimal thermal damage to other regions of the eye. Although a significant rise in temperature within the ocular lens was observed, the COMSOL model shows that the surrounding tissues, such as the posterior corneal tissues, would remain substantially undamaged due to the convective fluid flow of the aqueous humor. As expected, these effects were found to be dependent on the power of the laser. The simulations also demonstrated that pulsed operation of the laser resulted in temperature increases to smaller areas in the cataract, when compared to continuous wave operation. However, it was observed that the areas where temperature was elevated monotonically increased with pulse width. Given the larger pulse widths and probe size, we observed a higher temperature increase than published data seen in other femtosecond research. It should be noted, however, that our measurement is taken exactly at the point where the laser is exactly applied, compared to other research that use different reference points (e.g., lens surface).

Some notes may be in order. The simulation did not necessarily account for the movement of the probe around the optical lens which may under some circumstances distribute the thermal effects over a larger area. The entire lens was treated as a solid cataract, for simplicity of simulation. The simulation also did not address phase changes that may occur during surgery due to the ablation process creating a slurry-like solution inside the ocular lens. Notably, these simplifications do not obscure the answer to the question of whether millisecond pulses cause phacoemulsification of the cataract.

A prototype millisecond pulsed laser diode system for phacolysis was also constructed. One embodiment of the system included a laser diode configured to generate light in a millisecond pulsed regime; and an optical fiber element structured to be cooperated with the laser diode at a proximal end of the optical fiber element to receive the light from the laser diode. Such system, optionally, was characterized by one or more of the following features: (1) the light had a wavelength within an absorption band of water; (2) the light has an average power within a milliwatt range; (3) an output surface of a distal end of the optical fiber element was curved; and (D) the distal end of the optical fiber element was equipped with a termination configured to spatially converge light exiting from the distal end upon propagating of such light through the termination.

The operation of the system was tested on cataract-simulating gelatin samples. The multiple samples were categorized according to the clinical grades of cataracts seen in the operating room. It was proven that pulsed operation of the chosen laser diode at a wavelength specifically chosen to substantially coincide with the wavelength of strong light absorption at the eye tissue was able to constrain the thermal effects to a smaller area compared to continuous operation of laser. By constraining the thermal effects to a small area, a fragmentation pattern seen in conventionally performed clinical testing of phacoemulsification (see Mencucci, et al., Investigating the ocular temperature rise during femtosecond laser lens fragmentation: an in vitro study. Cataract, 2203-2210; 2015) was experimentally observed.

Finally, both the simulation and bench-top experiments used laser pulses with 50% duty cycle. This facilitated comparison of the effects of laser pulsing with the same average laser power across different pulse durations, and helped to ensure that the thermal cooling duration was not greater than the duration of the irradiation of the target cataract.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

Within this specification, embodiments have been described in a way that enables a clear and concise specification to be written, but it is intended and will be appreciated that embodiments may be variously combined or separated without parting from the scope of the invention. In particular, it will be appreciated that all features described herein are applicable to all aspects of the invention.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

Implementation of an embodiment of the invention-whether discussed expressly or not in this application-generally includes the use of electronic circuitry (for example, a computer processor) controlled by instructions stored in a memory, to perform specific data collection/processing and calculation steps as disclosed above. The memory may be random access memory (RAM), read-only memory (ROM), flash memory or any other memory, or combination thereof, suitable for storing control software or other instructions and data. Those skilled in the art should would readily appreciate that instructions or programs defining the operation of the present embodiment(s) may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement a method of the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

While the invention is described through the above-described exemplary embodiments, it will be understood by those of ordinary skill in the art that modifications to, and variations of, the illustrated embodiments may be made without departing from the inventive concepts disclosed herein.

The term “and/or”, as used in connection with a recitation involving an element A and an element B, covers embodiments having element A alone, element B alone, or elements A and B taken together.

While the invention is described through the above-described examples of embodiments, it will be understood by those of ordinary skill in the art that modifications to, and variations of, the illustrated embodiments may be made without departing from the inventive concepts disclosed herein. Disclosed aspects, or portions of these aspects, may be combined in ways not listed above. Accordingly, the invention should not be viewed as being limited to the disclosed embodiment(s).

1. A method comprising:
delivering light, generated by a laser diode source of light, to a target through a first medium surrounding the target while not interacting the light with the first medium along at least a first portion of an optical path of the light from the laser diode, said first portion traversing the first medium;
irradiating a target with the light, wherein said light configured such as to ensure that heating of the target, caused by the light, is substantially confined to an irradiated area of the target and not lost by thermal diffusion through the target; and
thermally emulsifying or liquifying the irradiated area of the target with the light.

2. A method according to claim 1, further comprising generating the light in a pulsed regime with a duration of a pulse being longer than a millisecond and an average power within a milliwatt range.

3. A method according to claim 1, wherein said delivering includes delivering said light through a second medium that is different from the first medium.

4. A method according to claim 1, wherein said delivering includes propagating said light along at least the first portion of the optical path while not changing a degree of divergence or a degree of convergence of the light during the propagating.

5. A method according to claim 1,
wherein, when the target includes a lens of an eye, said delivering includes delivering the light through the first medium along a path not intersecting an iris of the eye and/or
wherein, when the target includes the lens of the eye, said delivering includes delivering the light through the first medium along the path not substantially coinciding with the optical axis of the lens.

6. A method according to claim 5, wherein said delivering includes delivering the light through the first medium along the path substantially not intersecting the cornea of the eye.

7. A method according to claim 6, wherein said irradiating includes irradiating the target with a non-collimated beam of light.

8. A method according to claim 5, wherein said delivering excludes delivering along the path traversing aqueous humor.

9. A method according to claim 5, wherein, when the target includes a portion of the lens of an eye, each of said delivering, irradiating, and thermally ablating is carried out while not heating the cornea above 65 degrees Centigrade and/or while not heating the cornea above 60 degrees Centigrade.

10. A method according to claim 1, wherein said irradiating includes irradiating the target with a non-collimated beam of light.

11. A method according to claim 1, wherein, when the target includes a portion of the lens of an eye, each of said delivering, irradiating, and thermally ablating is carried out while not heating the cornea above 65 degrees Centigrade and/or while not heating the cornea above 60 degrees Centigrade.

12. A method according to claim 1, wherein said generating light includes generating light at a wavelength substantially overlapping with a spectral range corresponding to an absorption band of water.

13. A method according to claim 1, wherein said first portion of the optical path is a substantially straight linear portion.

14. A method according to claim 1, configured to carry out a laser phacoemulsification procedure with light pulses of millisecond duration.

15. An apparatus comprising:
a laser diode configured to generate light in a millisecond pulsed regime; and
an optical fiber element structured to be cooperated with the laser diode at a proximal end of the optical fiber element to receive the light from the laser diode,
wherein said apparatus is configured as a laser phacolysis apparatus.

16. An apparatus according to claim 15, wherein:
(16A) said light has a wavelength within an absorption band of water; and/or
(16B) said light has an average power within a milliwatt range; and/or
(16C) an output surface of a distal end of the optical fiber element is curved; and/or
(16D) the distal end of the optical fiber element is equipped with a termination configured to spatially converge light exiting from the distal end upon propagating of such light through the termination.

17. A method comprising:
performing a laser phacolysis with the use of the apparatus according to claim 15 by at least:
delivering the light, generated by the laser diode, to a target through a first medium surrounding the target while not interacting the light with the first medium along at least a first portion of an optical path of the light, said first portion traversing the first medium;
irradiating the target with the light, wherein said light is configured to ensure that heating of the target, caused by the light, is substantially confined to an irradiated area of the target and not thermally diffused through the target; and
thermally emulsifying or liquifying an area of the target irradiated with the light.

18. A method according to claim 17, wherein the performing comprises:
ablating at least a portion of the lens of an eye while not heating the cornea of the eye above 65 degrees Centigrade.

19. A method comprising:
carrying a laser phacolysis procedure without the use of a femtosecond laser source and/or without traversing laser light through the iris and/or without forming an incision at the cornea by:
delivering pulsed laser light with a millisecond pulse duration from a laser diode source to the lens of an eye through the sclera while not interacting said pulsed laser light with the sclera and with at least a portion of a humor body of the eye along a path of said delivering; and
ablating at least a portion of the lens of the eye while not heating the cornea above 65 degrees Centigrade.

20. A method according to claim 18, configuring said pulsed light, delivered to the lens, to ensure that heating of the lens, caused by the light, is substantially confined to an irradiated area of the lens and not lost by thermal diffusion through the target.
